(19) United States
(12) Patent Application Publication
(10) Pub. No.: US 2011/0071214 A1
(43) Pub. Date: Mar. 24, 2011

(54) METHODS AND COMPOSITIONS FOR THE TREATMENT OF CANCER

(76) Inventor: Gregory John Allen, Cockatoo (AU)

(21) Appl. No.: 12/992,620

(22) PCT Filed: May 13, 2009
(86) PCT No.: PCT/AU2009/000592

§ 371 Date: Nov. 15, 2010

Related U.S. Application Data
(62) Division of application No. 61/071,712, filed on May 14, 2008.

(51) Int. Cl. A61K 31/7088; C12N 15/63; A61P 35/00

(52) U.S. Cl. 514/44 R; 435/320.1

(57) ABSTRACT

The present invention provides compositions and methods for the treatment of cancer, and is predicated at least in part on the use of gene fusion regions as therapeutic targets. The fusion region target may be physically embodied at the level of DNA, RNA or protein. Typically the fusion region is specific to cancerous or precancerous cells, meaning that any therapy directed to the target may not significantly affect non-cancerous tissue. The fusion region may be targeted by protein or nucleic acid molecules capable of specifically binding to the region.

### FIELD OF THE INVENTION

The present invention pertains to the field of oncology. In particular, the invention is directed to the treatment of cancers demonstrating a gene fusion, such as leukemia, and some solid tumors such as endometrial cancer and sarcoma.

### BACKGROUND TO THE INVENTION

While the treatment of many cancers has undoubtedly advanced in recent years, it is generally accepted that many treatments are poorly tolerated. Many chemotherapeutic agents or other therapies (such as radiation therapy) have adverse effects on healthy tissue, often leading to intolerable side effects in patients. Poor side effect profiles leads to low patient compliance, and possibly even cessation of treatment.

Many anti-cancer treatments are also less efficacious than desired. While some cancers can sometimes be completely cured (such as breast cancer, bowel cancer, testicular cancer, skin cancer and Hodgkin lymphoma), the majority of cancers are never completely cleared and recur after cessation of therapy. The degree of success for any treatment depends on the individual, the particular cancer, and the chosen treatment regime.

It is therefore desirable to provide new cancer therapeutics having a better side effect profile and/or better efficacy than existing treatments. It is also desired to provide alternative treatments, to provide treatment options where a side effect is particularly problematic or the cancer particularly refractive. New therapeutics may also be useful as a part of combination therapies.

Cancers may be broadly divided into solid and non-solid cancers. Non-solid cancers include haematological malignancies such as leukemias. For the treatment of acute myelogenous leukemia (AML), the most common regimen involves 3 days of an anthracycline (e.g., daunorubicin, doxorubicin) and 7 days of ara-C. One option is chemotherapy with daunorubicin (Cerubidine®) or doxorubicin (Adriamycin®), plus cytarabine (ara-C; Cytosar-U®); also called “DA”. Observed side effects: Daunorubicin—myelosuppression (impaired bone marrow function), cardiotoxicity (heart damage), gastrointestinal effects; doxorubicin—cardiotoxicity, worsening of symptoms caused by other drugs; cytarabine—gastrointestinal effects (nausea, vomiting, diarrhoea), bleeding, and fever.

A further treatment option for AML is therapy with all-trans retinoic acid (ATRA). This is administered orally, however side effects include hyperleukocytosis (increased number of white blood cells); respiratory distress, fever, weight gain, edema, and pleural effusion.

For chronic myelogenous leukemia (CML), hydroxyurea (Hydrea®) is often used. It is normally administered orally on a 6-week trial, followed by treatment of indefinite length. Side effects include sore mouth, mouth ulceration, nausea, diarrhoea, rashes, bone marrow changes.

CML may also be treated with oral busulfan (Myleran®). The duration of treatment is usually 12-20 weeks. Side effects include myelosuppression (impaired bone marrow function), sterility in men and women, early menopause, skin pigmentation, cataracts, respiratory failure (“busulfan lung”).

For acute lymphocytic leukemia (ALL), chemotherapy usually begins with a three-drug schedule such as prednisone, vincristine sulfate (Oncovin®), and an anthracycline drug (e.g., daunorubicin). Prednisone is given orally in three divided doses, and Vincristine is given intravenously (IV). Prednisone and vincristine are given at weekly intervals for 4 weeks. Side effects of this regimen include hair loss and nervous system effects

Another option for ALL includes chemotherapy with prednisone, vincristine (Oncovin®), and L-asparaginase (Elspar®) or cyclophosphamide (Neosar®). Prednisone and vincristine are given at weekly intervals for 4 weeks; the schedule for L-asparaginase is more variable. Cyclophosphamide is given every 2 to 5 days, or by another schedule. Side effects include immune system effects, hair loss, nervous system effects, anaphylactic reaction to L-asparaginase, pancreatitis, blood clotting problems, infertility, severe bladder inflammation, cardiotoxicity, immune system suppression, and hair loss.

For ALL (and also CML), treatment with Imatinib mesylate (Glivec™, formerly STI571) can be preferred. This drug is a tyrosine kinase inhibitor selective for the ABL, KIT and PDGF-R kinases, and has shown considerable antileukemic activity.

Chemotherapy for chronic lymphocytic leukemia (CLL) often involves the use of chlorambucil (Leukeran®) or cyclophosphamide (Neosar®) plus prednisone, if needed. Chlorambucil is needed 4 days every month, and Cyclophosphamide every 2 to 5 days. Prednisone is given daily for 14 days, tapering off over 2 more weeks. Administration of Chlorambucil results in bone marrow toxicity; while side effects for cyclophosphamide include infertility, severe bladder inflammation, cardiotoxicity, immune system suppression, hair loss.

For Hairy Cell Leukemia (HCL), most newly diagnosed patients will receive chemotherapy with a purine analog. One option is the use of cladribine (2-chlorodeoxyadenosine; 2-CDA; Leustatin®), administered by continuous infusion intravenously (IV) over 7 days. Side effects include granulocytopenia, myelotoxicity, neurotoxicity, immunosuppression, fever, and infection

Another option for HCL involves the use of pentostatin (2-deoxycoformycin; “DCF”; Nipent®). This drug is administered as a bolus by IV, once every 14 days until maximum response is obtained. Similar side effects to those seen for cladribine are noted (see above).

A further problem with current treatments of leukemia is low efficacy. While some regimens may induce complete remission in younger patients, the high incidence of relapse results in low 3-year disease-free survival (DFS) rates. In patients failing first-line therapy, salvage chemotherapy has limited efficacy and rarely induces prolonged responses, despite considerable toxicity.

Non-chemotherapeutic approaches have also been used in the treatment of leukaemia. Allogenic stem cell transplantation (allo-SCT) is potentially curative, but treatment-related mortality and rate of disease recurrence are substantial. In patients undergoing SCT, 2-year overall survival (OS) of 17% (CR2/3) and 5% (PIF) have been reported, due to substantial transplant-related mortality and relapse. Overall, only a subset of patients actually undergoes allo-SCT because of older age, comorbidity, lack of a compatible donor or disease progression before SCT can be performed.

The treatment of solid tumours is also fought with difficulty with respect to side effects and efficacy. The treatment of solid tumors often relies, at least in part, on radiotherapy. External beam radiation therapy, treatment with high-energy rays or particles, may be used to kill cancer cells that remain in the tissues after surgery. The complications of external beam radiation therapy are swelling, sunburn-like skin changes in the treated area which can last for 6 to 12 months, fatigue and stomatitis. A further, albeit rare, complication is the development of another cancer called angiosarcoma. As for leukaemia, chemotherapy for solid tumors typically leads to significant side effects in the patient. Many chemotherapeutic agents lack sufficient selective toxicity, often leading to discontinuance of use due to intolerable side effects.

In light of the above, treatment for many cancers is problematic. It is an aspect of the present invention to overcome or ameliorate a problem of the prior art by proving alternative methods and compositions for the treatment of cancer.

A reference herein to a patent document or other matter which is given as prior art is not to be taken as an admission that that document or matter was, in Australia, known or that the information it contains was part of the common general knowledge as at the priority date of any of the claims.

### SUMMARY OF THE INVENTION

In a first aspect the present invention provides an expression vector comprising a promoter sequence operably linked to a sequence encoding a therapeutic protein, the therapeutic protein capable of binding to a fusion region present on a chimeric oncoprotein. In one embodiment the vector comprises a trafficking element, or is associated with a trafficking element. The trafficking element may be capable of directing the vector to a cancerous or pre-cancerous cell. In one embodiment, the trafficking element is capable of binding to a cell surface protein found on a cancerous or precancerous cell. In another embodiment, the trafficking element is capable of directing the vector to the nucleus of a cancerous or pre-cancerous cell. The trafficking element may comprise nuclear localization signal, or may be capable of directing the vector to the endoplasmic reticulum of a cancerous or pre-cancerous cell.

The fusion region may be found in a haematological cancer. The fusion region may comprises a BCR sequence, a ETV6 sequence, a RAR-alpha sequence, a MLL sequence, a AML1 sequence, a PDGFR-beta sequence, a FGFR1 sequence, a ALK sequence, a E2A sequence, a CBF-beta sequence, a ETO sequence, a FUS sequence, a DEK sequence, a HOXA9 sequence, a SET sequence, a BCM sequence, a REL sequence, a AF10 sequence, a MOZ sequence, a OTT sequence, a IG sequence.

In another embodiment of the vector the fusion region is found in a solid tumor cancer. The fusion region may comprise a EWS sequence, a ALK sequence, a RET sequence, a TRKA sequence, a SSX sequence, a PAX sequence, a CHOP sequence, a ASPL sequence.

In one form of the vector the therapeutic protein is a single-chained antibody.

The present invention further provides a pharmaceutical composition comprising a vector as described herein, and a pharmaceutically acceptable carrier. In one form of the invention the pharmaceutically acceptable carrier is a cationic agent such as poly-L-lysine. In another embodiment, the carrier comprises a trafficking element. In a particular form of the composition, the trafficking element is capable of directing the vector to a cancerous or pre-cancerous cell.

In another aspect the present invention provides a method for treating a cancer associated with a gene fusion, the method comprising the steps of administering to a subject in need thereof an effective amount of a ligand capable of binding to (i) a fusion region of a chimeric oncoprotein or (ii) a nucleic acid molecule encoding the fusion region, the fusion region being present in a cell of the subject. The fusion region may be found in a haematological cancer. The fusion region may comprises a BCR sequence, a ETV6 sequence, a RAR-alpha sequence, a MLL sequence, a AML1 sequence, a PDGFR-beta sequence, a FGFR1 sequence, a ALK sequence, a E2A sequence, a CBF-beta sequence, a ETO sequence, a FUS sequence, a DEK sequence, a HOXA9 sequence, a SET sequence, a BCM sequence, a REL sequence, a AF10 sequence, a MOZ sequence, a OTT sequence, a IG sequence.

In another embodiment of the vector the fusion region is found in a solid tumor cancer. The fusion region may comprise a EWS sequence, a ALK sequence, a RET sequence, a TRKA sequence, a SSX sequence, a PAX sequence, a CHOP sequence, a ASPL sequence.

In some embodiments, the method comprises the step of administering to the subject in need thereof an effective amount of a vector as described herein, or a composition as described herein.

In a further aspect the present invention provides a method of manufacturing a medicament including the use of an expression vector as described herein.

### DETAILED DESCRIPTION OF THE INVENTION

The present invention is predicated at least in part on Applicant's proposal that gene fusion regions which manifest in many cancerous and precancerous cells are useful as therapeutic targets. The fusion region target may be physically embodied at the level of DNA, RNA or protein. Typically the fusion region is specific to cancerous or precancerous cells, meaning that any therapy directed to the target may not significantly affect non-cancerous tissue. The fusion region may be targeted by protein or nucleic acid molecules capable of specifically binding to the region. Where the fusion region is embodied at the protein level, a protein ligand capable of specifically binding to the fusion region may be used to inhibit the biological activity or facilitate the degradation of the chimeric oncoprotein within the cell. Where the fusion region is embodied in DNA or RNA, an antisense ligand may be used to inhibit transcription or translation of the fused genes, resulting in a decrease in expression of the encoded chimeric oncoprotein. Without wishing to limited by theory, it is proposed that by lowering the level of a biologically active chimaeric oncoprotein (by inhibiting expression or altering activity), a cancerous or pre-cancerous cell will be prompted to differentiate. This may allow cells to resume a normal program of differentiation. By encouraging differentiation, the rate of proliferation may also decrease dramatically, as occurs with all-trans retinoic acid treatment in acute promyelocytic leukemia, and Imatinib (Glivec) treatment in chronic myelogenous leukemia.

The present invention is distinguished from the prior art which has hitherto recognised gene fusion regions as useful for the diagnosis and/or prognosis of certain cancers. For example, bone and soft tissue sarcomas of childhood demonstrate a range of gene fusions. Moreover, their characterization has revealed very consistent correlations between different gene fusion subtypes and the specific tumors that they are expressed in. Detection of fusion transcripts in pathologic specimens has therefore become useful as a diagnostic modality for these cancers. This is particularly relevant for childhood sarcomas, which tend to be extremely primitive in appearance and therefore very difficult to differentiate from each other morphologically. Since initial diagnosis often determines which treatment protocol a patient is entered on, accurate pathologic classification is a critical prognostic factor for these patients.

Thus, while the prior art has identified the usefulness of gene fusions as markers for the diagnosis and prognosis of cancer, their practical implementation as a therapeutic target has not previously been advanced. The present invention is therefore directed in one aspect to agents that are capable of decreasing the level of biologically active chimeric oncoproteins that are expressed from gene fusions in certain cancerous or precancerous cells.

Fusion occurs following illegitimate breakage and rejoining within the introns of both fusion partners, thereby maintaining coding integrity of the exons. A nonlimiting example of the fusion region found in the E2A/PBX1 chimeric oncoprotein:

Leu-Ser-Arg-Pro-Pro-Asp-Ser-Tyr-Ser-Gly-Leu-Gly-
Arg-Ala-Leu-Ser-Arg-Pro-Pro-Asp-Ser-Tyr-Ser-Tyr-
Ser-Val-Leu-Ser-Ile-Arg-Val-Leu-Cys-Glu-Ile-Lys-
Thr-Val-Leu-Ser-Ile-Arg

The residues in bold text are those derived from the E2A sequence, while those in normal text are derived from the PBX1 sequence. At the boundaries of coding exons both the 3′ end of the upstream codon and the 5′ end of the downstream codon contribute to the triplet which specifies the inter-exon aa. The break in the gene fusion region occurs in an intron. Thus the fusion protein joining aa, Val in the example, is specified by the 3′ end of E2A sequence and the 5′ end of the PBX1 sequence.

Applicant proposes that the amino acid sequence of the fusion region is relatively constant for the most frequently encountered chimaeric oncoproteins. However, where the fusion region is variable, the skilled person is enabled to sequence the oncoprotein to identify residues comprising the fusion region.

Accordingly, in a first aspect the present invention provides an expression vector comprising a promoter sequence operably linked to a sequence encoding a therapeutic protein, the therapeutic protein capable of binding to a fusion region present on a chimeric oncoprotein. Without wishing to be limited by theory, it is proposed that administration of the foregoing expression vector to a subject results in expression of the therapeutic protein within a cell of the subject. The therapeutic protein subsequently binds to a chimeric oncoprotein (if present) in the cell to form a complex. The complexed oncoprotein may then be neutralised by, for example, degradation within the endoplasmic reticulum of the cell. This mechanism has been validated by Richardson & Marasco (1995 Trends Biotechnol 13; 306, the contents of which is herein incorporated by reference). Single-chain antibodies, synthesized by the cell (that may also be targeted to a particular cellular compartment) can be used to interfere in a highly specific manner with cell growth and metabolism. Recent applications of this technology include the phenotypic knockout of growth-factor receptors, the functional inactivation of p21ras and the inhibition of HIV-1 replication.

In some cases, the therapeutic protein may not direct the degradation of the oncogenic protein, but may simply act to sterically inhibit binding of the oncogenic protein to another molecule such as an enzyme, nucleic molecule or receptor.

The skilled person is capable of identifying useful expression vectors relevant to the present invention. As will be appreciated, the expression vector must include sequences necessary to express protein in mammalian cells. For example, Promega Corporation (Madison, Wis., USA) supplies mammalian expression vectors in kit form under the names of CheckMate™/Flexi®, HaloTag™ pHT2, pACT, pAdVAntage™, pALTER®-MAX, pBIND pCAT®3, pCI, pCMVTNT™, pG5luc, phRG phRL, pSI, pTARGET™, and pTNT™. Invitrogen Corporation (Carlsbad, Calif., USA) supplies expression vector kits under the names of pcDNA3.1, pcDNA3.1-E, pcDNA4/HisMAX, pcDNA4/HisMAX-E, pcDNA3.1/Hygro, pcDNA3.1/Zeo, pZeoSV2, pRc/CMV2, pBudCE4, and pRc/RSV. The manufacturer's printed instructions included with the aforementioned mammalian expression vector kits are all incorporated herein by reference.

The expression vector comprises a sequence encoding a therapeutic protein, the therapeutic protein capable of binding to a fusion region present on a chimeric oncoprotein. The skilled person will be capable of designing or identifying an amino acid sequence capable of binding to any chimaeric oncoprotein. For example, a phage random peptide library approach may be used to identify amino acid sequences capable of binding to a chimeric oncoprotein. The skilled person is familiar with methods for screening such libraries to obtain one or more peptide sequences capable of binding to a chimeric oncoprotein. Phage display is a method that uses bacterial virus (phage) as a vehicle to express diverse protein or peptide sequences as part of the phage coat protein by cloning deoxyribonucleic acid (DNA) fragments in frame with phage coat protein genes. Upon viral infection, the expressed peptides are displayed on the surface of the viral particle. Depending on the DNA fragments cloned, phage display allows for expression of either short peptides or large proteins, such as immunoglobulin, on the viral surface. Phage display makes large-peptide diversity libraries readily attainable for identifying novel peptide ligands for protein targets. An exemplary phage display and screening method is described infra.

An alternative method for the development of active scFv's is to use the yeast 2 hybrid in vivo system, as described by Visintin et al 1999 Proc Natl Acad Sci USA vol 96 pp 11723-11728. VL and VH cDNAs, deriving from immunized mice are randomly linked using a linker encoding 15 amino acids (GGGGS) (3). Selection is performed using the yeast 2 hybrid in vivo system; positive candidate scFvs show binding activity against joining sequences of chimeric oncoproteins.

An alternative to the screening of random peptide libraries is to utilise amino acid sequences known in the prior art to bind to fusion regions of chimeric oncoproteins. For example, U.S. Pat. No. 699,917 (the contents of which is herein incorporated by reference) discloses monoclonal antibodies capable of specifically binding to the fusion region of the E2A/pbx1 oncoprotein that is seen in ALL. This document discloses a monoclonal antibody which specifically binds with an E2A/PBX1 fusion epitope. The monoclonal antibody will not specifically bind with an E2A peptide (PDSYS) or a PBX1 peptide (VLSIRGAQ), but will bind to the epitope produced from the fusion gene formed between E2A and PBX1. By very well known recombinant DNA techniques, the skilled person is capable of utilising sequences in the variable domain of antibodies disclosed in this patent document for the purposes of encoding a therapeutic protein in the present expression vectors. Alternatively, peptides may be designed de novo that are capable of binding to the chimeric oncoprotein.

In some embodiments, the present invention is further distinguished from the prior art by the inclusion of one or more trafficking elements associated with the vector. The trafficking elements are proposed to direct the vector to the cancerous or pre-cancerous cell, and/or to further direct the vector to the appropriate intracellular region or organelle within the cancerous or pre-cancerous cell. It is proposed that inclusion of trafficking elements will significantly increase the efficacy of treatment with the vectors as described herein.

In some forms of the invention, the trafficking element is capable of directing the vector to the nucleus of the cell. This may be accomplished via the covalent attachment of a a nuclear localization signal (NLS) to the vector. Prior art methods have failed to recognise the importance of the barrier provided by the nuclear membrane in transforming cells using an expression vector as described herein. Applicant proposes that significant improvements in the expression of molecules capable of binding to the fusion region of a chimeric oncoprotein are achieved by the use of a nuclear localization signal with the vectors described herein. The improvements in expression are proposed to provide more complete ablation of chimeric oncoproteins within the cell, leading to a higher efficacy in treating a cancer.

Methods for attaching a NLS to a DNA molecule have been previously disclosed, however not in the context of the present invention. An exemplary method is disclosed by Zanta et al (Proc Natl Acad Sci USA, 1999, Vol 96, 91-96; the contents of which is herein incorporated by reference). This method used a capped 3.3-kbp CMVLuciferase-NLS gene containing a single nuclear localization signal peptide (PKKKRKVEDPYC). Transfection of cells with the tagged gene remained effective down to nanogram amounts of DNA. Transfection enhancement (10- to 1,000-fold) as a result of the signal peptide was observed irrespective of the cationic vector or the cell type used. The reaction scheme for the chemical coupling steps leading to the oligonucleotide-peptide conjugate (oligo-NLS) is as follows. A hairpin oligonucleotide with a free alkylamino group in the T4 loop (oligo-NH2) was reacted with the heterobifunctional crosslinker SMCC to give a thiol-reactive maleimide oligonucleotide (oligo-Mal), which was in turn reacted with the C-terminal cysteinamide residue of the NLS dodecapeptide.

NLSs used to direct DNA into nuclei include SV40 T large antigen NLS-sequence 126PKKKRKV132, M9 a ‘nonclassical’ NLS rich in glycine and aromatic residues 268NQSSNFGPMKGGNFGGRSSGPYGGGGQYFAKPRNQGGY305) HIV-1 pre-integration complex and harbors a C-terminal, ‘nonclassical’ NLS (Vpr52-93, 52DTWTGVEALIRILQQLLFIHFRIGCRHSRIGIIQQRRTRNGA93) and the Ad3 fiber protein NLS (1AKRARLSTSFNPVYPYEDES20) (Cartier & Reszka 2002; Gene Therapy 9, 157).

While routine experimentation may demonstrate that certain NLS sequences are preferred over others for a given application, the skilled person understands that the present invention is not limited to the use of any particular NLS.

In another embodiment, the trafficking element is a ligand capable of binding to a cell surface molecule on the cancerous or pre-cancerous cell. It is proposed that efficacy is improved where the vector is delivered by way of interaction of the cell surface molecule with such ligand. It will be appreciated that it is not necessary for the ligand to be directed attached to the vector, but may be attached to a delivery vehicle associated with the vector. For example, the ligand may be covalently attached to a packaging protein such as poly-L-lysine, further details of which are presented infra.

In one embodiment, the ligand is capable of binding to the interleukin-3 (IL-3) receptor. This receptor is found on the surface of stem cells (including leukemic stem cells), and thus presents a means by which the vector may obtain entry into a cancerous or pre-cancerous cell. It is not important that non-leukemic cells may also express the IL-3 receptor protein because the ligand is not necessarily required for the purpose of selectively targeting leukemic cells. Selective toxicity is instead provided by the specific target sequence of the fusion region of the chimeric oncoprotein in the cancerous or pre-cancerous cell.

The IL-3 may be covalently attached to a polycationic agent (such as poly-L-lysine or poly(l-vinylimidazole)) in a complex of vector and polycationic agent. Compositions containing the vector in combination with poly-L-lysine are discussed elsewhere herein. Conjugation of the IL-3 to the polycation requires modification of 1-2 amino groups of IL-3 with the bifunctional reagent N-succinimydl-3-(2-pyridyldithio)propionate (SPDP). Similar modification of polycations of the heteroplex by SPDP allow conjugation through the formation of disulphide bonds (Cotten et al 1993, Methods in Enzymology 217, 618; the contents of which is herein incorporated by reference). Alternatively, bifunctional crosslinkers such as SMCC could be used.

A further embodiment provides that the trafficking element is capable of directing the therapeutic protein with bound chimeric oncoprotein to an organelle or region of the cell. In one embodiment, the vector comprises a DNA sequence capable of directing the therapeutic protein/chimeric oncoprotein complex to the endoplasmic reticulum, nucleus, a mitochondrion, a peroxisome, the plasma membrane the trans-Golgi network (TGN). Typically the trafficking element is introduced using an in frame fusion with a trafficking element peptide sequence.

The publication of Lo et al (2008, Handb Exp Pharmacol, 181, 343-73) generally discusses the subcellular localization of scFv molecules using signal peptides.

The vector may comprise any one or more trafficking elements, and in any combination.

The vector may further comprise a hairpin structure at one or both ends of the molecule. This may be achieved according to the method of Zanta et al (ibid). The hairpin structure at either end helps to protect the DNA from degradation. Hairpins are synthesized using an oligonucleotide synthesizer. One end includes a poly dT loop (5′-d(TCGATGTCCGCGTTGGCTTXTGCCAACGCGGACA) containing an amino-modified deoxythymidine (X; amino-modified dT, Glen Research, Sterling, Va.) and a SAL restriction site. This allows covalent attachment of the NLS (NH2-PKKKRKVEDPYC) to the DNA using the bifunctional crosslinker SMCC (4-(N-Maleimidomethyl)cyclohexane-1-carboxylic acid N-hydroxysuccinimide ester, from Sigma). The other hairpin end (5′-d(CCGGCTACCTTGCGAGCTTTTGCTCGCAAGGTAG), with an XMA restriction site, does not contain an NLS. The 2 different restriction sites on these hairpins allow directed ligation onto the ssAb DNA.

In one form of the vector the sequence encoding a therapeutic protein encodes a single-chain antibody (scFv). The scFV typically consists of immunoglobin heavy- and light-chain variable domains, covalently linked by a short, flexible polypeptide spacer; the size of these proteins is generally ˜28 kDa. Synthesis of appropriate single-chain antibodies may follow a scheme similar to that as outlined in Vaughan et al (1996 Nature Biotechnology 14; 309; the contents of which is herein incorporated by reference), or Marks et al (1991 J Mol Biol 222; 581; the contents of which is herein incorporated by reference). Alternatively, suitable clones i.e. those with avid binding to fused gene junctions, may be purchased commercially.

As an example of a representative method for producing and testing a cDNA construct encoding a scFv against the joining region of the chimeric oncoprotein AML1-ETO. A target amino acid sequence is selected within the joining sequence of AML1-ETO. The skilled person is capable of identifying a suitable target sequence. Firstly, for specificity the target sequence must encompass the fusion region. Secondly, the sequence must be of sufficient length to elicit an immune response. While routine experimentation may be used to identify a sufficient length, a peptide including at least 5 amino acid residues is typically required. A mouse is then immunized with the target sequence using standard techniques, and a scFv library is constructed using mRNA from the animal.

Synthesis of scFv DNA fragments may follow the general protocol of Clarkson et al 1991 (Nature 352; 624; the contents of which is herein incorporated by reference). This protocol directs the extraction of mRNA from the spleen of immunized mice, followed by 1st round cDNA synthesis. From this stage, the entire available VH and VL gene repertoire may be amplified by PCR in order to generate an scFv library. However it may be prefered to use gene segments from the VHIII/IGHV3 and VκI/IGVK1 immunoglobin gene families in scFv that are active within the reducing conditions found intracelluarly in eukaryotic cells. This approach may enrich for intracelluarly-active anti-joining region scFv's.

The VH and VL fragments are then joined together, using a short DNA linker that encodes the peptide sequence (gly4 ser)3, to make the complete scFv DNA.

Screening of the scFv library against the joining region of interest e.g. AML1-ETO, is performed using the yeast 2 hybrid method, a well known and commonly used protocol. The interaction between antibody and antigen in this method is adequately described in the prior art.

The avidity of the scFv to the chimeric oncogene is of relevance, since the greater the avidity the better the ablation of the oncogene. At this stage all positive clones may be tested for their avidity to the joining sequence. If the avidity of positive clones is less than that desired it may be necessary to expand the size of the library and screen for more potential candidates. Alternatively, it may be possible to use site directed mutagenesis to increase the avidity of a known positive candidate clone as described by Dona et al 2007 (BMC Cancer 7; 25).

For the purposes of testing, the scFv vector may be introduced to intoacute myeloblastic leukemia (i.e. AML1-ETO) leukemic stem cells in the form of long term culture initiating cells progenitors (LTC-ICs are CD34+, CD38−, CD71−, HLA-DR−, CD90−, CD117− and CD123+) to determine the progress of the scFv vector through endocytosis, cytoplasmic transport to the nucleus, transcription/translation and binding to the AML1-ETO oncoprotein.

The scFv vector may also be introduced into acute myeloblastic leukemia (i.e. AML1-ETO) leukemic stem cells in the form of long term culture initiating cells progenitors (LTC-ICs are CD34+, CD38−, CD71−, HLA-DR−, CD90−, CD117− and CD123+) to determine their affect upon growth and phenotype in semisolid agar.

Alternatively, the vector may be introduced into NOD SCID β microglobulin-deficient mice containing acute myeloblastic leukemia (i.e. AML1-ETO) leukemic stem cells (derived from bone marrow of affected patients) to assess the effect upon these leukemic mice, using standard haematological/histological methods.

In an exemplary method, the fusion region of the chimeric oncoprotein is first expressed in a bacterial system and subsequently purified. A glutathione S-transferase (GST) construct may be used. According to standard procedures well known to the skilled artisan, bacteria carrying the fusion region expression vector are grown and induced by isopropyl-β-D-thiogalactopyranoside. Resuspended bacteria are sonicated in radioimmunoprecipitation assay buffer [40 mmol/L HEPES (pH 7.4), 1% NP40, 0.1% SDS, 0.5% Na-deoxycholate (w/v), 150 mmol/L NaCl, 1 mmol/L phenylmethylsulfonyl fluoride, 10 μg/mL aprotinin, and 2 μg/mL leupeptin]. The fusion protein in the soluble fraction is then collected by glutathione-Sepharose 4B. Cleavage of the GST from the fusion protein is effected by factor Xa digestion. Reduced glutathione may also be added to an aliquot of the soluble fraction to release the fusion protein from the Sepharose 4B beads. The purified protein is then dialyzed against 1,000 mL of PBS (pH 7.4) at 4° C.

ScFv are then screened and isolated clones characterized. E. coli strain TG-1 [K12, (lac-pro), supE, thi, hsd5/F′traD36, proA+B+, lacIq, lacZM15] is used for the phage rescue. The nonsuppressor E. coli strain HB2151 [K12, ara, (lac-pro), thi/F′proA+B+, lacIqZM15] is used for the preparation of scFvs. are used (for example, Tomlinson's I and J Human scFv libraries, Medical Research Council Centre, Cambridge, United Kingdom). Both libraries are based on a single human framework for VH (V3-23/DP-47 and JH4b) and V (O12/O2/DPK9 and J1) with side chain diversity (DVT for TI and NNK for TJ encoded) incorporated at positions in the antigen binding site that makes contact to antigen in known co-crystal structures and highly diverse in the mature repertoire (18 different amino acid positions in total). The fusion protein is used as target for bio-panning. Libraries are preincubated with GST protein and supernatants subsequently applied to fusion protein-coated tubes to enrich for binders to recombinant fusion region. After a single round of selection, periplasmic extracts from individual clones are analyzed by indirect ELISA and specificity for the fusion region confirmed by competition ELISA. Binding of soluble scFv fragments is detected using horseradish peroxidase—conjugated rProtein L. Reactivity of phage and derived scFvs with recombinant and native chimeric oncoprotein, and GST, is determined by ELISA and immunoblotting.

The sequence of the scFv clone that provides the highest ELISA value, is amplified by PCR using primers containing appropriate restriction sites as well as sequences for the nuclear localization signal (5′-CCGGAATTCGCTGGATTGTTATTACTC-3′). The PCR product is cloned into the mammalian expression vector pIRES2-EGFP (Clontech) following the instructions of the manufacturer.

Methods exist in the art to express antibodies within the cell with a view to inactivating a specific protein. For example, genetic-selection technology (intracellular antibody capture) to facilitate the isolation of functional intracellular scFv from a diverse repertoire may be used. This approach comprises an in vitro library screen with scFv-expressing bacteriophage, employing bacterially expressed antigen, followed by a yeast in vivo antibody-antigen interaction screen of the sub-library of in vitro scFv antigen-binders. In the context of the present invention, the bacteriophage library could be screened against an oncogene fusion region, resulting in a scFv that is expressed intracellularly and capable of specifically binding to a chimeric oncoprotein (if present).

The method of Tewari et al (J. Immunol. 1998, 1;161(5):2642-2647; the contents of which is herein incorporated by reference) may be useful in the present invention. These workers targeted HIV p17 by intracellularly expressing a cDNA encoding an antibody to p17. cDNA from a hybridoma-secreting Ab to p17 was cloned, sequenced, reconstructed as a scFv, and expressed in the cytoplasm or nucleus with appropriate retention signals. The expressed scFvs bound specifically to HIV-1 p17, and inhibited viral replication.

Another potentially useful method is that of Richardson et al (Proc Natl Acad Sci USA, 1995, 92(8):3137-314; the contents of which is herein incorporated by reference) who inhibited the cell surface expression of the alpha subunit of the high-affinity interleukin 2 receptor (IL-2R alpha). A single-chain variable-region fragment of the anti-Tac monoclonal antibody was constructed with a signal peptide and a C-terminal ER retention signal. Intracellular expression of the single-chain antibody was found to completely abrogate cell surface expression of IL-2R alpha in stimulated Jurkat T cells. IL-2R alpha was detectable within the Jurkat cells as an immature 40-kDa form that was sensitive to endoglycosidase H, consistent with its retention in a pre- or early Golgi compartment. A single-chain antibody lacking the ER retention signal was also able to inhibit cell surface expression of IL-2R alpha although the mechanism appeared to involve rapid degradation of the receptor chain within the ER. These intracellular antibodies will provide a valuable tool for examining the role of IL-2R alpha in T-cell activation, IL-2 signal transduction, and the deregulated growth of leukemic cells which overexpress IL-2R alpha.

The prior art further discloses methods for the inhibition of expression of specific proteins using intracellular single-chain antibodies. For example, Sepp et al (J. Immunol. Methods, 1999, 231(1-2):191-205, the contents of which is herein incorporated by reference) disclose methods for generating a phenotypic knockout of the alpha1, 3Galactosyltransferase enzyme that is responsible using an intracellular antibody approach. The authors isolated high affinity anti-alpha1,3Galactosyltransferase single-chain antibodies from a semi-synthetic phage display library. Expression of a KDEL-tagged anti-alpha1,3Galactosyltransferase single-chain antibody in a porcine endothelial cell line resulted in the decreased expression of the Galalpha1-3Gal epitope.

The prior art discloses many other methods for generating specific intracellular antibodies that are capable of ablating the function of a protein in a cell, as described by Levy-Mintz et al (J. Virol. 1996, 70(12):8821-8832, the contents of which is herein incorporated by reference), Vetrugno et al (Biochem Biophys Res Commun, 2005, 338(4):1971-1977, the contents of which is herein incorporated by reference), Kitamura et al (J. Acquir. Immune Defic. Syndr. Hum. Retrovirol. 20(2):105-114, the contents of which is herein incorporated by reference), Kasono et al Biochem Biophys Res Commun 251(1):124-130, the contents of which is herein incorporated by reference), Jannot et al (Oncogene 1996, 13(2):275-282), Zhou et al (J Immunol, 160(3):1489-1496), Aires da Silva et al (J Mol Biol 340(3):525-542, the contents of which is herein incorporated by reference), Duan et al (Hum. Gene Ther. 5(11):1315-1324, the contents of which is herein incorporated by reference), Dauvillier et al (J Immunol 2002, 169(5):2274-22783, the contents of which is herein incorporated by reference), Poznansky et al (Hum Gene Ther 1999, 10(15):2505-2514, the contents of which is herein incorporated by reference), Yamamoto et al (Hepatology 1999, 30(1):300-307, the contents of which is herein incorporated by reference), and Piche et al (Gene Ther 1998, 5(9):1171-1179, the contents of which is herein incorporated by reference).

In another form of the vector, a nuclear localization signal is included. Upon delivery of the scFv cDNA to the cell, this attachment will increase the efficiency of cDNA transduction from the plasma membrane to the nucleus, thereby increasing the efficiency of expression. This may be achieved as follows: (a) a DNA fragment, containing a hairpin structure to which is added a nuclear localization signal, be synthesized. Experimental rationale and design for this procedure is from Zanta et al (1999 PNAS 96; 91; the contents of which is herein incorporated by reference) (b) this DNA/nuclear localization signal fragment is ligated to the scFv cDNA.

In one embodiment, only nucleosome incorporation is required, not chromosomal integration, since transcription can be episomal.

As discussed supra the level of biologically active chimeric oncoprotein may be lowered by decreasing expression of the oncoprotein. Methods are known in the art, including the use of small interfering RNA (siRNA) molecules, also known as short interfering RNA or silencing RNA. These molecules are 20-25 nucleotide-long double-stranded RNA molecules are involved in the RNA interference (RNAi) pathway where the siRNA interferes with the expression of a specific gene.

The skilled person is familiar with methods for the designing siRNA molecules. In the context of the present invention, it is necessary for the siRNA molecule to target the fusion region to ensure specificity of the siRNA. In one example, 21 nt sequences are identified in the target mRNA that begin with an AA dinucleotide. Beginning with the AUG start codon, the chimeric oncoprotein transcript is scaned for AA dinucleotide sequences. Each AA and the 3′ adjacent 19 nucleotides are recorded as potential siRNA target sites. This strategy for choosing siRNA target sites is based on the observation that siRNAs with 3′ overhanging UU dinucleotides are the most effective. This is also compatible with using RNA pol III to transcribe hairpin siRNAs because RNA pol III terminates transcription at 4-6 nucleotide poly(T) tracts creating RNA molecules with a short poly(U) tail.

siRNAs with other 3′ terminal dinucleotide overhangs have been shown to effectively induce RNAi. It is possible to modify this target site selection strategy to design siRNAs with other dinucleotide overhangs, but it is recommended that G residues in are avoided in the overhang because of the potential for the siRNA to be cleaved by RNase at single-stranded G residues.

2-4 target sequences are then selected. It has found that typically more than half of randomly designed siRNAs provide at least a 50% reduction in target mRNA levels and approximately 1 of 4 siRNAs provide a 75-95% reduction. Target sites are chosen from among the sequences identified identified above, based on the following guidelines:
siRNAs with 30-50% GC content are more active than those with a higher G/C content.
Since a 4-6 nucleotide poly(T) tract acts as a termination signal for RNA pol III, stretches of >4 T's or A's in the target sequence are to be avoided when designing sequences to be expressed from an RNA pol III promoter.
Since some regions of mRNA may be either highly structured or bound by regulatory proteins, it is preferable to select siRNA target sites at different positions along the length of the gene sequence.

The potential target sites are then compared to the appropriate genome database (human, mouse, rat, etc.) and any target sequences with more than 16-17 contiguous base pairs of homology to other coding sequences may be eliminated from consideration.

A complete siRNA experiment should include a number of controls to ensure the validity of the data. For example, a negative control siRNA with the same nucleotide composition as the selected siRNA but which lacks significant sequence homology to the genome. To design a negative control siRNA, the nucleotide sequence of the gene-specific siRNA are scrambled and a search conducted to ensure it lacks homology to any other gene.

Another control includes the use of additional siRNA sequences targeting the same mRNA. One way to improve confidence in RNAi data is to perform experiments, using a single siRNA at a time, with two or more different siRNAs targeting the same gene. Prior to these experiments, each siRNA should be tested to ensure that it reduces target gene expression by comparable levels.

In certain circumstances, the use of siRNA methods may be less efficacious than desired. It is proposed that protein-based methods (such as the use of intracellular antibodies) may provide more comprehensive ablation of chimeric oncoprotein activity in a cancerous or pre-cancerous cell. However, it will be understood that the present invention, at least in some embodiments extends to the use of molecules targeting nucleic acid, such as siRNA.

In a second aspect the present invention provides a method for treating a cancer associated with a gene fusion, the method comprising the steps of administering to a subject in need thereof an effective amount of a ligand capable of binding to (i) a fusion region of a chimeric oncoprotein or (ii) a nucleic acid molecule encoding the fusion region, the fusion region being present in a cell of the subject. As discussed supra a reduction in the level of biologically activity of a chimeric oncoprotein in a cell can be useful in the treatment of cancer. Targeting the therapy to the fusion region of the oncoprotein improves the selectivity of the method of treatment for cancerous or precancerous cells.

The step of administering an effective amount of a ligand capable of binding to a fusion region of a chimeric oncoprotein typically requires means for delivering the ligand to the cytoplasm of the cell of the subject. Where the ligand is a protein, this will generally be achieved by the delivery of an expression vector to the cytoplasm. Delivery may be achieved by one or more of any method known to the skilled person.

Delivery of the naked DNA is the simplest method of non-viral transfection. Clinical trials carried out of intramuscular injection of a naked DNA plasmid have occurred with some success. Levels of transformation may be improved using methods such as electroporation and the use of “gene gun” techniques, which delivers DNA coated gold particles into the cell using high pressure gas.

To improve the delivery of the new DNA into the cell, the DNA may be protected from damage and its entry into the cell must be facilitated. To this end new molecules, lipoplexes and polyplexes, have the ability to protect the DNA from undesirable degradation during the transfection process.

DNA may also be delivered with lipids in an organized structures such as micelles or liposomes. When the organized structure is complexed with DNA it is called a lipoplex. There are three types of lipids, anionic (negatively charged), neutral, or cationic (positively charged). Cationic lipids, due to their positive charge, were first used to condense negatively charged DNA molecules so as to facilitate the encapsulation of DNA into liposomes. Later it was found that the use of cationic lipids significantly enhanced the stability of lipoplexes. Also as a result of their charge, cationic liposomes interact with the cell membrane, endocytosis was widely believed as the major route by which cells uptake lipoplexes. Although cationic lipids are able to condense and encapsulate DNA into liposomes, the transfection efficiency can be improved by the use of “helper” lipids (usually electroneutral lipids, such as DOPE) to form lipoplexes. Relevant to the present invention, the most common use of lipoplexes has been in DNA transfer into cancer cells, where the supplied genes have activated tumor suppressor control genes in the cell and decreased the activity of oncogenes.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis, the process by which the polyplex enters the cell) such as inactivated adenovirus must occur. However, this isn't always the case, polymers such as polyethylenimine have their own method of endosome disruption as does chitosan and trimethylchitosan.

Hybrid methods have been developed that combine two or more techniques. Virosomes are one example; they combine liposomes with an inactivated HIV or influenza virus. This has been shown to have more efficient gene transfer in respiratory epithelial cells than either viral or liposomal methods alone. Other methods involve mixing other viral vectors with cationic lipids or hybridising viruses.

Dendrimers may also be used to facilitate the transformation of mammalian cells. A dendrimer is a highly branched macromolecule with a spherical shape. It is possible to construct a cationic dendrimer, i.e. one with a positive surface charge. When in the presence of genetic material such as DNA or RNA, charge complimentarity leads to a temporary association of the nucleic acid with the cationic dendrimer. On reaching the cell surface the dendrimer-nucleic acid complex is then taken into the cell via endocytosis. “Priostar” dendrimers (Dendritic Nanotechnologies, Michigan USA) can be specifically constructed to carry a DNA or RNA payload that transfects cells at a high efficiency with little or no toxicity.

In a particular form of the method, DNA is administered after condensation of the cDNA with poly L-lysine (90 to 450 lysine residues) (Cotton et al 1993 Methods Enzymology 217; 618, the contents of which is herein incorporated by reference). In this method the poly L-lysine condenses the DNA into small donut-shaped molecules.

Another form requires the covalent linkage of the DNA/poly L-lysine condensate with interleukin 3 (IL3). IL3 can direct delivery of the complex to leukemic stem cells as the IL3 receptor &#945; subunit (CD123) is commonly found on leukemic stem cells. It is envisaged that the cDNA/poly L-lysine/IL3 complex would enter the stem cells via receptor-mediated endocytosis utilizing the IL3 receptor. The method for covalent linkage of IL3 to poly L-lysine is modified from Cotton et al 1993 Methods Enzymology 217; 618. This strategy is considered particularly suitable for patient administration. Uptake by non-leukemic cells i.e. normal haematopoietic stem cells, is permitted since the activity of the scFv is only of consequence in leukemic cells.

In one embodiment of the method, the DNA is delivered by way of viral vector. The skilled artisan is familiar with methods for the delivery of heterologous DNA to a cell. Viruses are obligate intra-cellular parasites, designed through the course of evolution to infect cells, often with some specificity to a particular cell type. Viruses are typically very efficient at transfecting their own DNA into the host cell, which is expressed to produced new viral particles. By replacing genes that are needed for the replication phase of their life cycle (the non-essential genes) with foreign genes of interest, the recombinant viral vectors can transduce the cell type it would normally infect. To produce such recombinant viral vectors the non-essential genes are typically provided in trans, either integrated into the genome of the packaging cell line or on a plasmid. Though a number of viruses have been developed, most research has centred on four types; retroviruses (including lentiviruses), adenoviruses, adeno-associated viruses & herpes simplex virus type 1.

Retroviruses are a class of enveloped viruses containing a single stranded RNA molecule as the genome. Following infection, the viral genome is reverse transcribed into double stranded DNA, which integrates into the host genome & is expressed as proteins. The viral genome is approximately 10 kb, containing at least three genes: gag (coding for core proteins), pol (coding for reverse transcriptase) & env (coding for the viral envelope protein). At each end of the genome are long terminal repeats (LTRs) which include promoter/enhancer regions & sequences involved with integration. In addition there are sequences required for packaging the viral DNA (psi) & RNA splice sites in the env gene.

Retroviral vectors are most frequently based upon the Moloney murine leukaemia virus (Mo-MLV), which is an amphotrophic virus, capable of infecting both mouse cells, enabling vector development in mouse models, & human cells, enabling human treatment. The viral genes (gag, pol & env) are replaced with the transgene of interest and expressed on plasmids in the packaging cell line. Because the non-essential genes lack the packaging sequence (psi) they are generally not included in the virion particle. To prevent recombination resulting in replication competent retroviruses, all regions of homology with the vector backbone are typically removed the non-essential genes should be expressed by at least two transcriptional units.

The essential regions include the 5′&3′ LTRs and the packaging sequence lying downstream of the 5′ LTR. Transgene expression can either be driven by the promoter/enhancer region in the 5′ LTR, or by alternative viral (e.g. cytomegalovirus, Rous sarcoma virus) or cellular (e.g. beta actin, tyrosine) promoters. Mutational analysis has shown that up to the entire gag coding sequence and the immediate upstream region can be removed without effecting viral packaging or transgene expression. To aid identification of transformed cells selectable markers, such as neomycin & beta galactosidase, can be included and transgene expression can be improved with the addition of internal ribosome sites.

The retroviral envelope interacts with a specific cellular protein to determine the target cell range. Altering the env gene or its product has proved a successful means of manipulating the cell range. By replacing a portion of the env gene with 150 codons from the erythropoietin protein (EPO), it has been possible to target EPO receptor bearing cells with high affinity. Coupling an antibody to the viral particle with affinity for a second cell specific antibody via a streptavadin bridge, may also be used to improve viral uptake,

Viruses differ with respect to their tropisms, therefore by replacing the env gene with that of another virus, the host range can be extended, in a technique known as pseudotyping. Vesicular stomatitis virus G protein has been included in Mo-MLV derived vectors, which are also more stable when purified by ultracentrifugation. Improved transduction into numerous cell lines has been demonstrated by first treating the recipient cells with an adeno-associated vector expressing the cellular receptor for retroviral envelope protein.

For retroviral integration and expression of viral genes it is preferred that the target cells are dividing. The use of retroviral vectors may be advantageous in the context of the present invention, given that cancerous cells typically divide at a greater rate than non-cancerous cells.

Lentiviruses are a subclass of retroviruses which are able to infect both proliferating and non-proliferating cells. They are more complicated than simple retroviruses, containing an additional six proteins, tat, rev, vpr, vpu, nef & vif. Current packaging cell lines have separate plasmids for a pseudotype env gene, a transgene construct, and a packaging construct supplying the structural and regulatory genes in trans.

Adenoviruses are non-enveloped viruses containing a linear double stranded DNA genome. While there are over 40 serotype strains of adenovirus, most of which cause benign respiratory tract infections in humans, subgroup C serotypes 2 or 5 are predominantly used as vectors. The life cycle does not normally involve integration into the host genome, rather they replicate as episomal elements in the nucleus of the host cell and consequently there is no risk of insertional mutagenesis.

The wild type adenovirus genome is approximately 35 kb of which up to 30 kb can be replaced with foreign DNA. There are four early transcriptional units (E1, E2, E3 & E4), which have regulatory functions, and a late transcript, which codes for structural proteins. Progenitor vectors have either the E1 or E3 gene inactivated, with the missing gene being supplied in trans either by a helper virus, plasmid or integrated into a helper cell genome such as human fetal kidney cells, line 293. Second generation vectors additionally use an E2a temperature sensitive mutant. Some vectors contain only the inverted terminal repeats (ITRs) and a packaging sequence around the transgene, all the necessary viral genes being provided in trans by a helper virus. Adenoviral vectors are very efficient at transducing target cells in vitro and vivo, and may be produced to high titres (>10¹¹/ml).

Infection with a recombinant adenovirus can elicit an immune response in the patient. Approaches to avoid the immune response involving transient immunosupressive therapies have been successful in prolonging transgene expression and achieving secondary gene transfer. A less interventionist method has been to induce oral tolerance by feeding the host UV inactivated vector. However, it is desirable to manipulate the vector rather than the host. Although only replication deficient vectors are used, viral proteins are expressed at a very low level which are presented to the immune system. The development of vectors containing fewer genes, culminating in vectors which contain no viral coding sequences, has resulted in prolonged in vivo transgene expression in liver tissue.

The mechanism by which the adenovirus targeted the host cell is now understood. Uptake of the adenovirus particle has been shown to be a two stage process involving an initial interaction of a fibre coat protein in the adenovirus with a cellular receptor or receptors, which include the MHC class I molecule and the coxsackievirus-adenovirus receptor. The penton base protein of the adenovirus particle then binds to the integrin family of cell surface heterodimers allowing internalisation via receptor mediated endocytosis. Most cells express primary receptors for the adenovirus fibre coat protein, however internalisation is more selective. Methods of increasing viral uptake include stimulating the target cells to express an appropriate integrin, and conjugating an antibody with specificity for the target cell type to the adenovirus (Wickham et al, 1997b, Goldman et al, 1997). By incorporating receptor binding motifs into the fibre coat protein, it is possible to redirect the virus to bind the integrin expressed by damaged endothelial or smooth muscle cells, or heparin sulphate receptors which is expressed by numerous cells types.

Adeno-associated viruses (AAV) are non-pathogenic human parvoviruses, dependant on a helper virus, usually adenovirus, to proliferate. They are capable of infecting both dividing and non dividing cells, and in the absence of a helper virus integrate into a specific point of the host genome (19q 13-qter) at a high frequency. The wild type genome is a single stranded DNA molecule, consisting of two genes; rep, coding for proteins which control viral replication, structural gene expression and integration into the host genome, and cap, which codes for capsid structural proteins. At either end of the genome is a 145 bp terminal repeat (TR), containing a promoter:

When used as a vector, the rep & cap genes are replaced by the transgene and associated regulatory sequences. Production of the recombinant vector requires that rep & cap are provided in trans, along with helper virus gene products (E1a, E1b, E2a, E4 & VA RNA from the adenovirus genome). The conventional method is to cotransfect two plasmids, one for the vector and another for rep and cap, into 293 cells infected with adenovirus. Another protocol removes all adenoviral structural genes and use rep resistant plasmids or conjugate a rep expression plasmid to the mature virus prior to infection.

In the absence of rep, the AAV vector will only integrate at random, as a single provirus or head to tail concatamers, once the terminal repeats have been slightly degraded. Interest in AAV vectors has been due to their integration into the host genome allowing prolonged transgene expression. Gene transfer into many cell types has been reported, with prolonged expression often noted. Neutralising antibody to the AAV capsid may be detectable, but does not prevent readministration of the vector or shut down promoter activity.

Herpes simplex virus type 1 (HSV-1) is a human neurotropic virus. The wild type HSV-1 virus is able to infect neurones and either proceed into a lytic life cycle or persist as an intranuclear episome in a latent state. Latently infected neurones function normally and are not rejected by the immune system. Although the latent virus is transcriptionally almost silent, it does possess neurone specific promoters that are capable of functioning during latency.

The viral genome is a linear double stranded DNA molecule of 152 kb. There are two unique regions, long and short (termed UL & US) which are linked in either orientation by internal repeat sequences (IRL & IRS). At the non-linker end of the unique regions are terminal repeats (TRL & TRS). There are up to 81 genes, of which about half are not essential for growth in cell culture. Once these non essential genes have been deleted, 40-50 kb of foreign DNA can be accommodated within the virus. Three main classes of HSV-1 genes have been identified, namely the immediate-early (IE or alpha) genes, early (E or beta) genes & late (L or gamma) genes.

Gene expression during latency is driven by the latency associated transcripts (LATs) located in the IRL region of the genome. Two LATs (2.0 & 1.5kb) are transcribed in the opposite direction to the IE gene ICP0. LATs have a role in HSV-1 reactivation from latency and the establishment of latency. Two latency active promoters which drive expression of the LATs have been identified and may prove useful for vector transgene expression.

Two basic approaches have been used for production of HSV-1 vectors, namely amplicons & recombinant HSV-1 viruses. Amplicons are bacterially produced plasmids containing col E1 ori (an Escherishia coli origin of replication), OriS (the HSV-1 origin of replication), HSV-1 packaging sequence, the transgene under control of an immediate-early promoter & a selectable marker. The amplicon is transfected into a cell line containing a helper virus (a temperature sensitive mutant) which provides all the missing structural & regulatory genes in trans. Both the helper and amplicon containing viral particles are delivered to the recipient. More recent amplicons include an Epstein-Barr virus derived sequence for plasmid episomal maintenance.

Recombinant viruses are made replication deficient by deletion of one the immediate-early genes e.g. ICP4, which is provided in trans. Although they are less pathogenic and can direct transgene expression in brain tissue, they are toxic to neurones in culture. Deletion of a number of immediate-early genes substantially reduces cytotoxicity and also allows expression from promoters that would be silenced in the wild type latent virus. These promoters may be of use in directing long term gene expression.

Viral vectors according to the present invention may comprise one or more of trafficking elements as described herein. For example, the capsid protein of a vrial vector may be engineered to express a protein capable of binding to a cell surface molecule on the cancerous or precancerous cell.

As required by the present methods of treatment, the cancer is associated with a gene fusion. The majority of acute leukemias arise as a consequence of a gene fusion. Typical fusions include BCR fusions (for example, BCR-ABL, BCR/FGFR1, BCR/JAK2, BCR/PDGFRα), ETV6 fusions (for example ETV6/ABL, ETV6/JAK2, ETV6-PDGFR□, ETV6-SYK, ETV6-ARG, ETV6-TRKC, ETV6-FGFR3, ETV6-CDX2, ETV6-AML1, ETV6-MN1), RARα fusions (for example RARα-PML, RARα-NPM, RARα-NuMA, RARα-PLZF, RARα-STAT5b), MLL fusions (for example AF4-MLL, AF9-MLL, AF10-MLL, MLL-ENL, AFX1-MLL, AF1P-MLL, AF6-MLL, MLL-AF17,. internal duplications of MLL, deletions of MLL), AML1 fusions (for example AML1-ETV6, AML1-ETO, AML1-CBFA2T3, AML1-EVI/EAP, AML1-FOG2), PDGFRβ fusions (for example PDGFRβ-ETV6, PDGFRβ-HIP1, PDGFRβ-RABEP1 (Rabaptin), PDGFRβ-(H4)/CCDC6, PDGFRβ-TPM3, PDGFRβ-PDE4DIP, PDGFRβ-PRKG2, PDGFRβ-GPIAP1, PDGFRβ-GIT2, PDGFRβ-NIN, PDGFRβ-KIAA1509, PDGFRβ-TP53BP1, PDGFRβ-NDE1, and PDGFRβ-SPECC1 (HCMOGT-1)), FGFR1 fusions (for example FGFR1-FIM (ZNF198 or RAMP), FGFR1-FOP, FGFR1-CEP110, FGFR1-FGFR10P2), ALK fusions (for example ALK-ALO17, ALK-MYH9, ALK-MSN, ALK-ATIC, ALK-NPM, ALK-CLTC, ALK-TFG, ALK-TPM3, ALK-TPM4), and other fusions such as E2A-PBX1, E2A-HLF, CBFβ-MYH11, ETO-AML1, FUS-ERG, DEK-CAN, DEK-NUP214, HOXA9-NUP98, SET-CAN, BCM-IL2, REL-NRG, AF10-CALM, MOZ-CBP, MOZ-TIF2, MOZ-p300, OTT-MAL, and IG-BCL6

The treatment of leukemic stem cells is particularly advantageous because (i) they are responsible for the generation of the entire cell population of the leukemia and (ii) being stem cells they can enter a G₀ state of quiescence and evade the normal anti-cancer protocols that are designed specifically against proliferating cells. Accordingly, the present invention may be capable of targeting the cells responsible for leukemia at first instance, these cells being overlooked in prior art protocols.

Some gynaecological cancers also show involvement of gene fusions. For example, endometrial stromal tumors including benign stromal nodules, low-grade endometrial stromal sarcomas (ESS), and undifferentiated endometrial sarcomas (UES) often demonstrate a gene fusion on chromosome 7 that includes two zinc-finger genes (JAZF1 and JJAZ1). For example, it has been demonstrated that the JAZF1/JJAZ1 fusion transcript occurred in 80% of analyzed ESS cases.

Gene fusions are also noted in various sarcomas. For example, synovial sarcomas display a characteristic SYT-SSX fusion gene resulting from the chromosomal translocation. This fusion has been detected in virtually all synovial sarcomas. The translocation fuses the SYT gene from chromosome 18 to either of two highly homologous genes at Xp11, SSX1 or SSX2. SYT-SSX1 and SYT-SSX2 are thought to function as aberrant transcriptional regulators.

Malignant melanoma is another serious cancer in which gene fusion is demonstrable. The genes involved in the translocation are found recurrently in malignant melanoma of soft parts have been characterized and shown to form hybrid transcripts. The deduced chimeric protein encoded by the der(22) chromosome consists of the N-terminal domain of EWS linked to the bZIP domain of ATF-1, a transcription factor which may normally be regulated by cAMP. ATF-1 has not previously been implicated in oncogenesis. EWS was first identified as forming a hybrid transcript in Ewing's sarcoma, which links its N-terminal domain to the DNA binding domain of the FLI-1 gene. Thus the oncogenic conversion of EWS follows a common scheme of activation, exchanging its putative RNA binding domain with different DNA binding domains that appear to be tumour-specific.

Studies have shown that glandular tumors such as mucoepidermoid carcinomas (MECs) of the salivary and bronchial glands are characterized by a recurrent translocation, resulting in a MECT1-MAML2 fusion in which the cAMP response element binding protein (CREB)-binding domain of the CREB-regulated transcriptional coactivator MECT1 (also known as CRTC1, WAMTP1, or TORC1) is fused to the transactivation domain of the Notch coactivator MAML2. Other glandular tumors demonstrating gene fusions include Warthin's tumor (WAT) and clear cell hidradenoma of the skin.

A subset of renal carcinomas is associated with translocations resulting TFE3 gene fusions (PRCC-TFE3, PSF-TFE3, NONO-TFE3, ASPL-TFE3), encoding related aberrant transcription factors. Studies have reported the cloning of a novel clathrin heavy-chain gene (CLTC)-TFE3 gene fusion resulting from a translocation in a renal carcinoma. The fusion transcript joined the 5′ exons of CLTC on chromosome band 17q23 to the 3′ exons of TFE3.

Further fusions associated with solid tumors include EWS fusions (for example EWS-FLI, EWS-ERG, EWS-ETV1, EWS-ATF1, EWS-CHN, EWS-WT1), ALK fusions (for example ALK-CLTC, ALK-TFG, ALK-TPM3, ALK-TPM4, ALK-RANBP2, ALK-CARS, ALK-SEC31L1), and other such as RET-PTC1/PTC3, RET-PTC2, TRKA-TPM3, TRKA-TPR, TRKA-TPM3, SSX1/SSX2-SYT, PAX3-FKHR, PAX7-FKHR, CHOP-TLS/FUS and ASPL-TFE3

From the foregoing, it will be apparent that gene fusions are present in many types of cancer. However, it is to be appreciated that the present invention is not restricted to the particular fusions described herein. Other fusions that have not yet been identified are nonetheless included within the scope of the present invention.

In one embodiment of the method, the ligand is delivered by an expression vector as described herein. The dosage regime will be determined by the physician by reference to parameters such as patient history, height, weight, surface area, prognosis, tumor burden, and the like. The dosage may also be determined by reference to animal studies. In any event, routine experimentation may be used to determine an efficacious dose for an individual. By commencing dosage at a low dose, and titrating the dosage upwards while monitoring the patient for relevant clinical signs and symptoms it will be possible to empirically arrive at an effective dose that has an acceptable side effect profile.

In another aspect the present invention further provides a method of manufacturing a medicament including the use of an expression vector described herein.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed

1. An expression vector comprising a promoter sequence operably linked to a sequence encoding a therapeutic protein, the therapeutic protein capable of binding to a fusion region present on a chimeric oncoprotein.

2. A vector according to claim 1 comprising a trafficking element, or associated with a trafficking element.

3. A vector according to claim 2 wherein the trafficking element is capable of directing the vector to a cancerous or pre-cancerous cell.

4. A vector according to claim 3 wherein the trafficking element is capable of binding to a cell surface protein found on a cancerous or precancerous cell.

5. A vector according to claim 2 wherein the trafficking element is capable of directing the vector to the nucleus of a cancerous or pre-cancerous cell.

6. A vector according to claim 5 wherein the trafficking element is a nuclear localization signal.

7. A vector according to claim 2 wherein the trafficking element is capable of directing the vector to the endoplasmic reticulum of a cancerous or precancerous cell.

8. A vector according to claim 1 wherein the fusion region is found in a haematological cancer.

9-79. (canceled)

80. A vector according to claim 8 wherein the fusion region comprises a sequence selected from the group consisting of a BCR sequence, an ETV6 sequence, a RAR-alpha sequence, a MLL sequence, a AML1 sequence, a PDGFR-beta sequence, a FGFR1 sequence, a ALK sequence, an E2A sequence, a CBF-beta sequence, a ETO sequence, a FUS sequence, a DEK sequence, a HOXA9 sequence, a SET sequence, a BCM sequence, a REL sequence, a AF10 sequence, a MOZ sequence, a OTT sequence, and a IG sequence.

81. A vector according to claim 1 wherein the fusion region is found in a solid tumor cancer.

82. A vector according to claim 8 wherein the fusion region comprises sequence selected from the group consisting of a EWS sequence, a ALK sequence, a RET sequence, a TRKA sequence, a SSX sequence, a PAX sequence, a CHOP sequence, and a ASPL sequence.

83. A vector according to claim 1 wherein the therapeutic protein is a single-chained antibody.

84. A pharmaceutical composition comprising a vector according to claim 1, and a pharmaceutically acceptable carrier.

85. A composition according to claim 84 wherein the carrier comprises a trafficking element capable of directing the vector to a cancerous or pre-cancerous cell.

86. A method for treating a cancer associated with a gene fusion, the method comprising the steps of administering to a subject in need thereof an effective amount of a ligand capable of binding to (i) a fusion region of a chimeric oncoprotein or (ii) a nucleic acid molecule encoding the fusion region, the fusion region being present in a cell of the subject.

87. A method according to claim 86, the method comprising the step of administering to the subject in need thereof an effective amount of a vector according to claim 1.

88. A method according to claim 86 wherein the fusion region is found in a haematological cancer.

89. A method according to claim 88 wherein the fusion region comprises a sequence selected from the group consisting of a BCR sequence, an ETV6 sequence, a RAR-alpha sequence, a MLL sequence, a AML1 sequence, a PDGFR-beta sequence, a FGFR1 sequence, a ALK sequence, an E2A sequence, a CBF-beta sequence, a ETO sequence, a FUS sequence, a DEK sequence, a HOXA9 sequence, a SET sequence, a BCM sequence, a REL sequence, a AF10 sequence, a MOZ sequence, a OTT sequence, and a IG sequence.

90. A method according to claim 86 wherein the fusion region is found in a solid tumor cancer.

91. A method according to claim 90 wherein the fusion region comprises a sequence selected from the group consisting of a EWS sequence, a ALK sequence, a RET sequence, a TRKA sequence, a SSX sequence, a PAX sequence, a CHOP sequence, and a ASPL sequence.

92. A method according to claim 86, the method comprising the step of administering to the subject in need thereof an effective amount of a composition according to claim 84.
